(19) United States
(12) Patent Application Publication
(10) Pub. No.: US 2019/0038688 A1
(43) Pub. Date: Feb. 7, 2019

(54) COMPOSITIONS AND METHODS FOR MOOD STABILIZATION

(71) Applicant: Wake Up Sense, LLC, Ogden, UT (US)

(72) Inventor: Michael J. Gonzalez, Ponce, PR (US)

(21) Appl. No.: 15/495,717

(22) Filed: Apr. 24, 2017

(51) Int. Cl. A61K 36/185; A61K 36/16; A61K 31/405; A61K 31/20; A61K 31/661; A61K 31/194; A61K 31/519; A61K 31/198; A61K 31/555; A61K 9/00; A61K 31/51; A61K 31/525; A61K 31/455; A61K 31/197; A61K 31/675; A61K 31/4188; A61K 31/714; A61K 31/375; A61K 31/593; A61P 25/24; A23L 33/175; A23L 33/12; A23L 33/105; A23L 33/16; A23L 33/155

(52) U.S. Cl. CPC A61K 36/185; A23V 2002/00; A61K 31/405; A61K 31/20; A61K 31/661; A61K 31/194; A61K 31/519; A61K 31/198; A61K 31/555; A61K 9/0053; A61K 31/51; A61K 31/525; A61K 31/455; A61K 31/197; A61K 31/675; A61K 31/4188; A61K 31/714; A61K 31/375; A61K 31/593; A61P 25/24; A23L 33/175; A23L 33/12; A23L 33/105; A23L 33/16; A23L 33/155; A61K 36/16

(57) ABSTRACT

The present disclosure provides compositions which contain 5-hydroxytryptophan (5-HTP), omega-3 fatty acids, St. John's Wort (hypericum perforatum, 3% hyperacin), Inositol Hexaphospate, Inositol Bitartrate, and Ginkgo biloba in combination with magnesium citrate, 5-Methylfolate, L-Theanine, and zinc picolinate in therapeutically effective amounts for providing mood stabilization or a decrease in depression in patients, as well as methods of using and manufacturing such compositions.

### CROSS-REFERENCE TO RELATED APPLICATION

This application claims the benefit of U.S. Provisional Application No. 62/326,305, filed Apr. 22, 2016, the disclosure of which is incorporated herein by reference in its entirety.

### BACKGROUND

Compositions containing non-pharmaceutical nutritional ingredients that have individually been shown to have a positive effect on mood when included in a person's diet have been shown to have beneficial effects in mood improvement and stabilization. However, ordinary nutritional supplements, such as those commonly available in health food stores pharmacies and supermarkets, are typically intended as prophylactic doses. Such supplements may only temporarily alter serum nutritional levels and are not usually capable of therapeutic action to effectively reduce symptoms or severity of depression and/or anxiety.

Past experimental data demonstrated that daily administration of a combination supplement that was formerly offered by applicant could significantly decrease depression scores on standard measures. The components of this known combination supplement formula are 5-hydroxytryptophan (5-HTP), omega-3 fatty acids, St. John's Wort, Inositol Hexaphospate, Inositol Bitartrate, and Ginkgo biloba, all in a proprietary blend with some other vitamins and minerals.

Despite this positive result, improvements that included additional ingredients that provided for more significant effects would be an improvement in the art.

### SUMMARY

The present disclosure provides compositions which contain 5-hydroxytryptophan (5-HTP), omega-3 fatty acids, St. John's Wort (hypericum perforatum, 3% hyperacin), Inositol Hexaphospate, Inositol Bitartrate, and Ginkgo biloba, magnesium citrate, 5-Methylfolate, L-Theanine, and zinc picolinate in therapeutically effective amounts for providing mood stabilization or a decrease in depression in patients, as well as methods of using and manufacturing such compositions. While the earlier formulation discussed in the background was found to have clinically significant results, it was discovered by the inventor that the inclusion of the additional ingredients provided an enhanced effect which provides better results and may allow for reduced amounts of supplements used by a patient.

Embodiments of compositions in accordance with the present disclosure when properly used in accordance with the present disclosure will change a subject's biochemistry at a cellular level by substantially increasing the intake of coenzyme and cofactor nutrients by the cells. The inclusion of 5-Methylfolate, and its proportion to the other ingredients, allows it to act as a carrying agent on a cellular level which is optimized to safely enhance intracellular nutritional levels by carrying nutrients in the compositions in accordance with the present disclosure to the center of the cell where it is really required to achieve the desired results through metabolic correction to intervene with impaired biochemical reactions, achieving synergistic effects.

### DETAILED DESCRIPTION

The present disclosure is directed to compositions for mood stabilization and methods of formulating, manufacturing and using compositions for mood stabilization. It will be appreciated by those skilled in the art that the embodiments herein described, while illustrative, are not intended to so limit this disclosure or the scope of the appended claims. Those skilled in the art will also understand that various combinations or modifications of the embodiments presented herein can be made without departing from the scope of this disclosure. All such alternate embodiments are within the scope of the present disclosure.

Metabolic Correction is a functional term introduced by Dr. Michael J. Gonzalez and Dr. Jorge R. Miranda-Massari in 2011 to explain the mechanism of how nutrients can correct biochemical disruptions that promote the disease state by improving cellular biochemistry to help the body achieve metabolic or physiological optimization. In general, metabolic correction is a biochemical physiological approach that favors metabolic harmony as a requisite to optimal health, and that vitamins, minerals and nutrients in this context function as cofactors in the orchestration of enzymatically-driven biochemical reactions, and can be harnessed towards the improvement of cellular biochemistry to ultimately achieve metabolic/physiological optimization, primarily via the fact that increased concentrations of these agents fundamentally improve enzymatic/metabolic efficiency, which in turn may repair genetic and hereditary defects and disorders consequent to defective enzymes, allowing for: (1) reduction of drug dosing/scheduling requirements, (2) reduction of adverse events, and (3) improvement of patient outcome. The methods and formulas of the present disclosure are designed to use metabolic correction to achieve the desired results, in some cases removing the need for individuals using them to continue to use anti-depressant medications.

Metabolic Correction intervenes with impaired biochemical reactions that are associated with a lack of wellbeing. In other words, Metabolic Correction is a fine-tuning of the cellular physiology to improve function, therefore preserving health, preventing tissue damage, and reverting disease.

We must eat a wide variety of food to obtain the substances that we need. A big problem that we face is that the nutritional value of foods that people eat seems to be greatly inferior to the listed values given in food tables. A study looking into this issue showed declines in protein of 6%; calcium, 16%; phosphorus, 9%; iron, 15%; riboflavin, 38%; and vitamin C, 20%. There is a dilution effect, in which yield-enhancing methods such as fertilization and irrigation may decrease nutrient concentrations, an environmental dilution effect. Recently, evidence has emerged that genetically based increases in yield may have the same result, a genetic dilution effect. Modern crops that grow larger and faster cannot necessarily acquire nutrients at the same, faster rate, whether by synthesis or from the soil. Today's foods are not as nutritious as those eaten in the past. US and UK governmental statistics show a decline in trace minerals of up to 76% in fruit and vegetables over the period 1940 to 1991. The nutritional decline findings alone give reason to eat organic fruits and vegetables. In fact, for nearly all nutrients, organic fruits and vegetables remain the most nutrient-dense foods. This information makes the updated food pyramid not so much current as reflective of the need for an increase in fruits and vegetables in order to get the same nutritional benefits. Also, Americans on average do not even come close to the recommendations to limit added sugars, refined carbohydrates, and added fats and oils.

Additionally, more than 100,000 deaths are reported annually in the U.S. due to medication properly prescribed and taken as directed. The incidence of serious and fatal adverse side effects in US hospitals is extremely high; these are frequent and more so than generally recognized. Fatal adverse side effects appear to be the fourth leading cause of death in the US. If medication is necessary, providing Metabolic Correction principles may reduce medication requirements, reduce adverse side effects, and improve therapeutic outcome.

Metabolic Correction principles may also be used to help compensate for the increased demand of nutrients due to a disease state Burns lead to loss of protein and essential nutrients. Surgery increases the need for zinc, vitamin C, and other nutrients involved in cellular-tissue repair. Broken bones need calcium, magnesium, and vitamin C for healing. Infections challenge the immune system and place high demand on nutritional resources such as zinc, B-complex vitamins, and vitamin C. The same nutritional demand is present when one is exposed to chemical, physical, and emotional stress. Chronic disease sufferers are at higher risk of interaction of drugs and nutrients. There are thousands of conceivable genetic defects (inborn or acquired), so it is likely that many people have higher genetic requirements for many micronutrients. We need a better understanding of the interrelationship between nutritional biochemistry and the disease-pathological state.

The majority of the chemical reactions that take place in living organisms are catalyzed by enzymes. The mechanisms of enzyme-catalyzed reactions in general involve (1) the formation of a complex between the enzyme and a substrate and (2) the breakdown of this complex to form the product of the reaction. The rate determining step is usually the breakdown of the complex to form the product. Under conditions such that the concentration of the complex corresponds to equilibrium with the enzyme and the substrate, the rate of the reaction is given by the Michaelis-Menten equation.

The rate of an enzyme-catalyzed reaction is approximately proportional to the concentration of the reactant, until concentrations that largely saturate the enzyme are reached. The saturating concentration is larger for a defective enzyme with decreased combining power for the substrate than for the normal enzyme. For such a defective enzyme, the catalyzed reaction could be made to take place at or near its normal rate by an increase in the substrate concentration. This mechanism of action of gene mutation is only one of several that lead to disadvantageous manifestations that could be overcome by an increase in the concentration of enzymatic cofactors. These binding problems may result in metabolic inefficiency with the accumulation of metabolic byproducts. In general, this is the law of mass action: as the vitamin and mineral concentration increases, enzyme efficiency increases. These considerations obviously suggest a rationale for Metabolic Correction wherein you provide the required cofactors in the amount needed to improve function. This increased enzyme efficiency may allow a genetic defect to be overcome. This biochemical activity follows the chemical principle of Le Chatlier, which states that when stress is applied in an equilibrium situation, it will move in the direction that minimizes stress. In this case there is an unfavorable equilibrium of active enzyme that with the addition of the necessary nutrients will be moved toward a more physiologically favorable metabolic state.

Many human genetic diseases due to defective enzymes can be remedied or ameliorated by the administration of high doses of the vitamin component of the corresponding coenzyme, which can partially restore the enzymatic activity. Several single nucleotide polymorphisms in which the variant amino acid reduces coenzyme binding and thus enzymatic activity can be remedied by raising cellular concentrations of the cofactor through high-dose nutrient therapy.

Inadequate intakes of vitamins and minerals from food can lead to DNA damage, mitochondrial decay, and other pathologies. Ames suggests that evolutionary allocation of scarce micronutrients by enzyme triage is an explanation for why DNA damage is commonly found in micronutrient deficiency. Motulsky has also argued that many of the common degenerative diseases are the result of imbalanced nutritional intake with genetically determined needs.

As an example, folic acid and vitamin B12 have an important function in the maintenance of nuclear and mitochondrial genome integrity. Both in vivo and in vitro studies with human cells show that deficiency of these vitamins causes an array of problems in the nuclear and mitochondrial DNA that can be minimized with increased folate and cobalamin concentrations. In order to acquire the protective effect of these vitamins, they are needed in concentrations that are obtained at intake levels above the current recommended dietary intakes of folate (>400 μg/day) and vitamin B12 (>2μ/day).

Chromosome breaks lead to mutations that precede tissue damage and disease. Many types of physiological impairments due to inadequacy of vitamins and minerals can lead to suboptimal organ-system function including poor drug metabolism, insufficient neurotransmitter production, and impaired immune defenses. Chronic vitamin-mineral undernutrition reduces immune competency and central nervous system efficiency, while increasing morbidity, which may lead to increases in degenerative diseases. This approach to optimizing health by improving enzyme efficiency and thereby metabolism and physiology is the basis of Metabolic Correction.

An example of Metabolic Correction is that high-dose B vitamins can counteract a poor Km. As many as one-third of mutations in a gene result in the corresponding enzyme having an increased Km (decreased binding affinity) for a coenzyme, causing a lower rate of reaction. About 50 different human genetic diseases due to a poorer binding affinity of the mutant enzyme for its coenzyme can be remedied by feeding high-dose B vitamins, which raise levels of the corresponding coenzyme; many polymorphisms also result in a lowered affinity of enzyme for its coenzyme and thus may be in part remediable.

To summarize, Metabolic Correction has two important biological actions: (1) optimization of cellular function by improving enzymatic efficiency and (2) producing a pharmacological effect to correct abnormal cell function due to a biochemical disarray occasioned by the disease process.

An optimum intake of micronutrients and metabolites, which varies with age, environmental factors, and genetics, should tune up metabolism and markedly increase health at a modest cost, particularly for the poor, obese, and elderly.

In one illustrative embodiment, the composition may include a propriety blend of 5-hydroxytryptophan (5-HTP), omega-3 fatty acids, St. John's Wort (hypericum perforatum, 3% hyperacin), Inositol Hexaphospate, Inositol Bitartrate, and Ginkgo biloba (24% ginkgo flavone glycocide), magnesium citrate, 5-Methylfolate, L-Theanine, and zinc picolinate in therapeutically effective amounts for providing mood stabilization or a decrease in depression in patients.

In some illustrative embodiments, these ingredients may be present in a composition in accordance with the present disclosure in the following proportions 5-Methylfolate in about 1 part to about 2 parts, 5-hydroxytryptophan (5-HTP) about 1,000 parts, omega-3 fatty acids about 10,000 parts, St. John's Wort (hypericum perforatum, 3% hyperacin) about 6,000 parts, Inositol Hexaphospate about 1,000 parts, Inositol Bitartrate about 1,000 parts, and Ginkgo biloba (24% ginkgo flavone glycocide) about 800 parts, magnesium citrate about 2,000 parts to about 10,000 parts, L-Theanine about 500 parts, and zinc picolinate about 200 parts. It is noted that Inositol Bitartrate and Choline bitrartrate are considered equivalents and either may be used.

The inclusion of 5-Methylfolate, and its proportion to the other ingredients, allows it to act as a carrying agent on a cellular level which is optimized to safely enhance intracellular nutritional levels by carrying nutrients in the compositions in accordance with the present disclosure to the center of the cell where it is really required to achieve the desired results through metabolic correction to intervene with impaired biochemical reactions, and achieving synergistic effects.

Additional ingredients may include vitamin B1 (for example, in the form of thiamine pyrophosphate), vitamin B2 (for example, in the form of riboflavin 5-phosphate), vitamin B3 (for example, in the form of niacinamide and/or nicotinic acid), vitamin B5 (for example, in the form of a pantothenic acid salt, such as calcium-pantothenate), vitamin B6 (for example, in the form of pyridoxal 5-phosphate), vitamin B7, vitamin B12 (for example, in the form of methylcobalamin), vitamin C (as ascorbic or citric acid), and/or vitamin D3.

These additional ingredients may be present in the compositions in the following proportions vitamin B1 about 1,000 parts, vitamin B2 about 1,000 parts, vitamin B3 about 1,000 parts, vitamin B5 about 1,000 parts, vitamin B6 about 1,000 parts, vitamin B7 about 5 parts, vitamin B12 about 2 parts, vitamin C about 5,000 parts, and/or vitamin D3 about 0.8 parts.

The compositions may also include one or more minerals, such as calcium, iron, magnesium, and selenium.

In further formulation of the compositions, various ingredients may be included in the compositions to improve the aesthetic qualities of the compositions, such as taste, color and ingestibility. Such ingredients would include, for example, flavorings, colorants, binders, fillers, flow agents and lubricating agents. These agents are well-known in the pharmaceutical and dietary supplement industries for formulating compositions into various forms, including tablets, capsules, powders and the like.

One illustrative example of an exemplar formula for a composition in accordance with the present disclosure may comprise the following:

| Ingredient | Form | Amount | |
|---|---|---|---|
| 5-HTP | 5-hydroxytryptophan 40% PE | 50 | mg |
| Omega 3 Fatty Acids | | 500 | mg |
| St. John's Wort | hypericum perforatum 0.3% | 300 | mg |
| | Hyperacin | | |
| Vitamin B1 | thiamine pyrophosphate | 50 | mg |
| Vitamin B2 | riboflavin 5-phosphate | 50 | mg |
| Vitamin B3 | niacinamide | 25 | mg |
| Vitamin B3 (Niacin) | Nicotinic Acid | 25 | mg |
| Vitamin B5 | pantothenic acid(calcium- | 50 | mg |
| | pantothenate) | | |
| Vitamin B6 | Pyridoxal 5-phosphate | 50 | mg |
| Vitamin B7 | biotin (USP) | 250 | mcg |
| Vitamin B9 | folic acid (methylfolate) | 50 | mcg |
| Vitamin B12 | methylcobalamin | 100 | mcg |
| Vitamin C | | 250 | mg |
| Vitamin D3 | | 40 mcg = | |
| | | 400 IU | |
| Ginkgo Biloba | 24% ginkgo flavone glycocide | 40 | mg |
| Magnesium | Citrate | 500 | mg |
| Inositol | Hexaphosphate | 50 | mg |
| Choline | bitrartrate USP | 50 | mg |
| L-Theanine | | 25 | mg |
| Zinc | picolinate | 10 | mg |

A second illustrative example of an exemplar formula for a composition in accordance with the present disclosure may comprise the following:

| Ingredient | Form | Amount | |
|---|---|---|---|
| 5-HTP | 5-hydroxytryptophan 40% PE | 50 | mg |
| Omega 3 Fatty Acids | | 500 | mg |
| St. John's Wort | hypericum perforatum 0.3% | 300 | mg |
| | Hyperacin | | |
| Vitamin B1 | thiamine pyrophosphate | 50 | mg |
| Vitamin B2 | riboflavin 5-phosphate | 50 | mg |
| Vitamin B3 | niacinamide | 25 | mg |
| Vitamin B3 (Niacin) | Nicotinic Acid | 25 | mg |
| Vitamin B5 | pantothenic acid(calcium- | 50 | mg |
| | pantothenate) | | |
| Vitamin B6 | Pyridoxal 5-phosphate | 50 | mg |
| Vitamin B7 | biotin (USP) | 250 | mcg |
| Vitamin B9 | folic acid (methylfolate) | 100 | mcg |
| Vitamin B12 | methylcobalamin | 100 | mcg |
| Vitamin C | | 250 | mg |
| Vitamin D3 | | 40 mcg = | |
| | | 400 IU | |
| Ginkgo Biloba | 24% ginkgo flavone glycocide | 40 | mg |
| Magnesium | Citrate | 100 | mg |
| Inositol | Hexaphosphate | 50 | mg |
| Choline | bitrartrate USP | 50 | mg |
| L-Theanine | | 25 | mg |
| Zinc | picolinate | 10 | mg |

The foregoing exemplar formulations are not intended to limit the compositions of the present disclosure. Other ingredients may be added to, and indeed may be eliminated from, the general formula set forth above and still be within the scope of the claimed invention. It is requisite to the invention, however, to provide a therapeutically effective amount of the blend of 5-hydroxytryptophan (5-HTP), omega-3 fatty acids, St. John's Wort (hypericum perforatum, 3% hyperacin), Inositol Hexaphospate, Inositol Bitartrate, and Ginkgo biloba, magnesium citrate, 5-Methylfolate, L-Theanine, and zinc picolinate.

Although the foregoing exemplar compositions may be formed into a tablet form, capsule forms and powdered forms are also within the scope of the invention and are within the skill in the art to form by known methods. However, by way of example, tablets comprising the ingredients noted above may be formulated by mixing the ingredients, all of which are in a dry powder form, in a ribbon blender until well blended. The resulting mixture is then compressed to a specified weight (e.g., 2,000 mg), a specified thickness (e.g., 0.135 inches) and specified hardness (e.g., 1118-25 Kp) using a suitable tablet press.

Tablets or capsules of the compositions of the present disclosure may further be formed or formulated for timed- or delayed-release by, for example, providing a suitable outer or intermediary coating on or in the tablet or capsule, in known manner, to provide a selected time release of the composition. Any suitable means known in the art by which tablets and capsules may be formed or formulated for timed- or delayed-release may be used.

As previously noted, the compositions of the present disclosure are particularly suitable for use in mood stabilization. For humans, a daily dosage of the compositions containing a therapeutically effective amount of the required blend may be in the form of capsules having amounts similar to those set forth in the exemplar formulas above. The daily dosage may be administered as three such capsules, taken at three different times during the day, or as a larger dose taken at a single time with time release properties.

Embodiments of compositions in accordance with the present disclosure when properly used in accordance with the present disclosure will change a subject's biochemistry at a cellular level by substantially increasing the intake of coenzyme and cofactor nutrients by the cells. The inclusion of 5-Methylfolate and its proportion to the other ingredients allows it to act as a carrying agent which is optimized to safely enhance intracellular nutritional levels, by carrying nutrients in the compositions in accordance with the present disclosure to the center of the cell where it is really required.

The formulas and constituent ingredients described herein are merely examples of various formulations for the compositions of the present invention and are not intended to limit the scope of the invention. Those of skill in the art will understand that the compositions of the present invention, in addition to providing the requisite therapeutically effective amount of the required blend as described herein, may include other ingredients to facilitate the formulation of the compositions.

### EXAMPLE 1

A first group of patients are randomly selected from a base group of patients diagnosed with a depressive disorder by a psychiatrist based on DSM-V and who are not taking any medication or dietary supplements except for those associated with depression status.

The first group of selected patients receives a three-capsule dosage of the first exemplar formula on a daily basis for thirty days.

The Beck Depression Inventory, a physical activity questionnaire, a dietary recall questionnaire, and anthropometric indices are completed in the beginning and end of the study.

At the end of the study, Beck scores are significantly lower for the first group, indicating a reduction in depression.

### EXAMPLE 2

A first group of patients and a second group of patients are randomly selected from a base group of patients diagnosed with a depressive disorder by a psychiatrist based on DSM-V and who are not taking any medication or dietary supplements except for those associated with depression status.

The first group of selected patients receives a three-capsule dosage of the second exemplar formula on a daily basis for thirty days. The second group receives a placebo on the same dosing schedule.

The Beck Depression Inventory, a physical activity questionnaire, a dietary recall questionnaire, and anthropometric indices are completed in the beginning and end of the study.

At the end of the study, Beck scores are lower for all members of the first group and as a group are significantly lower than the scores for the second group of patients, indicating an increased reduction in depression in comparison to the second group.

### EXAMPLE 3

A first group of patients and a second group of patients are randomly selected from a base group of patients diagnosed with a depressive disorder by a psychiatrist based on DSM-V and who are not taking any medication or dietary supplements except for those associated with depression status.

The first group of selected patients receives a three-capsule dosage of the second exemplar formula on a daily basis for thirty days. The second group receives a combination supplement containing 5-hydroxytryptophan (5-HTP), omega-3 fatty acids, St. John's Wort (hypericum perforatum, 3% hyperacin), Inositol Hexaphospate, Inositol Bitartrate, and Ginkgo biloba, but without the remaining ingredients on the same dosing schedule.

The Beck Depression Inventory, a physical activity questionnaire, a dietary recall questionnaire, and anthropometric indices are completed in the beginning and end of the study.

At the end of the study, Beck scores are lower for all members of the first group and the second group and are significantly lower for both groups as a whole. However, the scores of the first groups are significantly lower than the scores for the second group of patients, indicating an increased reduction in depression in the first group in comparison to the second group.

While this disclosure has been described using certain embodiments, it can be further modified while keeping within its spirit and scope. This application is therefore intended to cover any variations, uses, or adaptations of the disclosure using its general principles. Further, this application is intended to cover such departures from the present disclosure as come within known or customary practices in the art to which it pertains and which fall within the limits of the appended claims.

What is claimed is:

1. A method for stabilizing moods or decreasing depression in humans comprising administering to a human by oral administration a nutritional supplement comprising a blend of therapeutically effective amounts of:
5-hydroxytryptophan (5-HTP);
omega-3 fatty acids;
St. John's Wort (hypericum perforatum, 3% hyperacin);
Inositol Hexaphospate;
Inositol Bitartrate or Choline bitrartrate;
Ginkgo biloba;
magnesium citrate;
5-Methylfolate;
L-Theanine; and
zinc picolinate.

2. The method of claim 1, wherein administering to a human by oral administration a nutritional supplement comprises administering a nutritional supplement wherein the 5-hydroxytryptophan (5-HTP); omega-3 fatty acids; St. John's Wort (hypericum perforatum, 3% hyperacin); Inositol Hexaphospate; Inositol Bitartrate or Choline bitrartrate; Ginkgo biloba, magnesium citrate; 5-Methylfolate; L-Theanine; and zinc picolinate are in a ratio a:b:c:d:e:f:g:h:i:j, respectively such that the a as 5-hydroxytryptophan (5-HTP) is present in a an amount of about 1,000, the b as omega-3 fatty acids is present in an amount of about 10,000 parts, the c as St. John's Wort (hypericum perforatum, 3% hyperacin) is present in an amount of about 6,000 parts, the d as Inositol Hexaphospate is present in an amount of about 1,000 parts, the e as Inositol Bitartrate or Choline bitrartrate is present in an amount of 1,000 parts, the f as Ginkgo biloba contains about 24% ginkgo flavone glycocide and is present in an amount of about 800 parts, the g as magnesium citrate is present in an amount of about 2,000 parts to about 10,000 parts, the h as 5-Methylfolate is present in an amount of about 1 part to about 2 parts, the i as L-Theanine is present in an amount of about 500 parts, and the j as zinc picolinate is present in an amount of about 200 parts.

3. The method of claim 2, further comprising forming the nutritional supplement composition into a dosage unit form for oral administration wherein the 5-hydroxytryptophan (5-HTP) is present in an amount of about 50 mg, the omega-3 fatty acids are present in an amount of about 500 mg, the St. John's Wort (hypericum perforatum, 3% hyperacin) is present in an amount of about 300 mg, the Inositol Hexaphospate is present in an amount of about 50 mg; the Inositol Bitartrate or Choline bitrartrate is present in an amount of about 50 mg, the Ginkgo biloba is present in an amount of about 40 mg, the magnesium citrate is present in an amount of from about 100 mg to about 500 mg, the 5-Methylfolate is present in an amount of from about 50 mcg to about 100 mcg, the L-Theanine is present in an amount of about 25 mg, and the zinc picolinate is present in an amount of about 10 mg.

4. The method of claim 2, wherein the administering to a human by oral administration comprises administering at least one dosage unit at least three times over the course of a day.

5. The method of claim 2, wherein the nutritional supplement further comprises vitamin B1, vitamin B2, vitamin B3, vitamin B5, vitamin B6, vitamin B7, vitamin B12, vitamin C, and vitamin D3.

6. The method of claim 5, wherein the vitamin B1 comprises thiamine pyrophosphate, the vitamin B2 comprises riboflavin 5-phosphate, the vitamin B3 comprises niacinamide and/or nicotinic acid, the vitamin B5 comprises a pantothenic acid salt, the vitamin B6 comprises pyridoxal 5-phosphate, the vitamin B12 comprises methylcobalamin, and the vitamin C comprises ascorbic or citric acid.

7. The method of claim 5, wherein the vitamin B1 is present in an amount of about 1,000 parts, the vitamin B2 is present in an amount of about 1,000 parts, the vitamin B3 is present in an amount of about 1,000 parts, the vitamin B5 is present in an amount of about 1,000 parts, the vitamin B6 is present in an amount of about 1,000 parts, the vitamin B7 is present in an amount of about 5 parts, the vitamin B12 is present in an amount of about 2 parts, the vitamin C is present in an amount of about 5,000 parts, and the vitamin D3 is present in an amount of about 0.8 parts.

8. The method of claim 7, wherein the nutritional supplement composition is provided as a dosage unit form for oral administration wherein the vitamin B1 is present in an amount of about 50 mg, the vitamin B2 is present in an amount of about 50 mg, the vitamin B3 is present in an amount of about 50 mg, the vitamin B5 is present in an amount of about 50 mg, the vitamin B6 is present in an amount of about 50 mg, the vitamin B7 is present in an amount of about 250 mcg, the vitamin B12 is present in an amount of about 100 mcg, the vitamin C is present in an amount of about 250 mg, and the vitamin D3 is present in an amount of about 40 mcg.

9. The method of claim 1, wherein administering a nutritional supplement comprises administering a supplement containing one or more minerals.

10. The method of claim 9, wherein the supplement contains at least one mineral selected from the list comprising calcium, iron, magnesium, and selenium.

11. A nutritional supplement composition for stabilizing moods or decreasing depression in humans comprising:
5-hydroxytryptophan (5-HTP);
omega-3 fatty acids;
St. John's Wort (hypericum perforatum, 3% hyperacin);
Inositol Hexaphospate;
Inositol Bitartrate or Choline bitrartrate;
Ginkgo biloba;
magnesium citrate;
5-Methylfolate;
L-Theanine; and
zinc picolinate.

12. The nutritional supplement composition of claim 11, wherein the 5-hydroxytryptophan (5-HTP); omega-3 fatty acids; St. John's Wort (hypericum perforatum, 3% hyperacin); Inositol Hexaphospate; Inositol Bitartrate or Choline bitrartrate; Ginkgo biloba; magnesium citrate; 5-Methylfolate; L-Theanine; and zinc picolinate are in a ratio a:b:c:d:e:f:g:h:i:j, respectively such that the a as 5-hydroxytryptophan (5-HTP) is present in an amount of about 1,000, the b as omega-3 fatty acids is present in an amount of about 10,000 parts, the c as St. John's Wort (hypericum perforatum, 3% hyperacin) is present in an amount of about 6,000 parts, the d as Inositol Hexaphospate is present in an amount of about 1,000 parts, the e as Inositol Bitartrate or Choline bitrartrate is present in an amount of 1,000 parts, the f as Ginkgo biloba contains about 24% ginkgo flavone glycocide and is present as in an amount of about 800 parts, the g as magnesium citrate is present in an amount of about 2,000 parts to about 10,000 parts, the h as 5-Methylfolate is present in an amount of about 1 part to about 2 parts, the i as L-Theanine is present in an amount of about 500 parts, and the j as zinc picolinate is present in an amount of about 200 parts.

13. The nutritional supplement composition of claim 12, wherein the nutritional supplement composition is in a dosage unit form for oral administration to a human and wherein the 5-hydroxytryptophan (5-HTP) is present in an amount of about 50 mg, the omega-3 fatty acids are present in an amount of about 500 mg, the St. John's Wort (hypericum perforatum, 3% hyperacin) is present in an amount of about 300 mg, the Inositol Hexaphospate is present in an amount of about 50 mg; the Inositol Bitartrate or Choline bitrartrate is present in an amount of about 50 mg, the Ginkgo biloba is present in an amount of about 40 mg, the magnesium citrate is present in an amount of from about 100 mg to about 500 mg, the 5-Methylfolate is present in an amount of from about 50 mcg to about 100 mcg, the L-Theanine is present in an amount of about 25 mg, and the zinc picolinate is present in an amount of about 10 mg.

14. The nutritional supplement composition of claim 13, wherein the nutritional supplement composition is in a dosage unit form for oral administration to a human in the form of a tablet or capsule.

15. The nutritional supplement composition of claim 14, wherein the dosage unit form for oral administration to a human in the form of a tablet or capsule further comprises a tablet or capsule formulated for delayed-release to provide a selected time release of the composition.

16. The nutritional supplement composition of claim 12, further comprising vitamin B1, vitamin B2, vitamin B3, vitamin B5, vitamin B6, vitamin B7, vitamin B12, vitamin C, and vitamin D3.

17. The nutritional supplement composition of claim 16, wherein the vitamin B1 comprises thiamine pyrophosphate, the vitamin B2 comprises riboflavin 5-phosphate, the vitamin B3 comprises niacinamide and/or nicotinic acid, the vitamin B5 comprises a pantothenic acid salt, the vitamin B6 comprises pyridoxal 5-phosphate, the vitamin B12 comprises methylcobalamin, and the vitamin C comprises ascorbic or citric acid.

18. The nutritional supplement composition of claim 16, wherein the vitamin B1 is present in an amount of about 1,000 parts, the vitamin B2 is present in an amount of about 1,000 parts, the vitamin B3 is present in an amount of about 1,000 parts, the vitamin B5 is present in an amount of about 1,000 parts, the vitamin B6 is present in an amount of about 1,000 parts, the vitamin B7 is present in an amount of about 5 parts, the vitamin B12 is present in an amount of about 2 parts, the vitamin C is present in an amount of about 5,000 parts, and the vitamin D3 is present in an amount of about 0.8 parts.

19. The nutritional supplement composition of claim 18, wherein the nutritional supplement composition is in a dosage unit form for oral administration to a human and wherein the vitamin B1 is present in an amount of about 50 mg, the vitamin B2 is present in an amount of about 50 mg, the vitamin B3 is present in an amount of about 50 mg, the vitamin B5 is present in an amount of about 50 mg, the vitamin B6 is present in an amount of about 50 mg, the vitamin B7 is present in an amount of about 250 mcg, the vitamin B12 is present in an amount of about 100 mcg, the vitamin C is present in an amount of about 250 mg, and the vitamin D3 is present in an amount of about 40 mcg.

20. The nutritional supplement composition of claim 12, further comprising at least one mineral selected from the list comprising calcium, iron, magnesium, and selenium.
